(19) United States
(12) Patent Application Publication
(10) Pub. No.: US 2026/0294648 A1
(43) Pub. Date: Oct. 1, 2026

(54) SURGICAL PLANNING METHOD AND CHARACTERIZATIONS FOR IMPROVED OUTCOMES

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventors: Elizabeth A. DUXBURY, Andover, MA (US); Nathan A. NETRAVALI, Andover, MA (US); Steven YURICK, Pittsburgh, PA (US); Mara C. PALMER, Pittsburgh, PA (US); Riddhit MITRA, Pittsburgh, PA (US)

(21) Appl. No.: 19/475,999

(22) PCT Filed: May 13, 2024
(86) PCT No.: PCT/US2024/029066

§ 371 Date: Oct. 16, 2025

Related U.S. Application Data
(60) Provisional application No. 63/465,941, filed on May 12, 2023.

(51) Int. Cl. A61F 2/46; A61B 34/10; A61B 34/30; G16H 20/40

(52) U.S. Cl. CPC A61F 2/461; A61B 34/30; G16H 20/40; A61B 2034/105; A61B 2034/108; A61F 2002/4633; A61F 2002/4668

(57) ABSTRACT

Systems and methods for planning a robotic-assisted total knee arthroplasty are disclosed herein. A method may include receiving surface data corresponding to boney anatomy of a knee. A three-dimensional model may be generated based on the surface data. The method may include generating three-dimensional Coronal Plane Alignment of the Knee (CPAK) classification data based on the three-dimensional model and determining an optimal implant selection and implant position based on the three-dimensional CPAK classification data.

The present disclosure relates generally to methods and systems for improving outcomes in Total Knee Arthroplasty (TKA) procedures.

### BACKGROUND

Total Knee Arthroplasty (TKA) is a surgical procedure commonly used to relieve pain and restore function in patients suffering from knee joint diseases such as osteoarthritis, rheumatoid arthritis, and post-traumatic arthritis. The procedure involves replacing the damaged or diseased knee joint with an artificial joint, or implant. The success of a TKA procedure largely depends on the accurate selection and positioning of the implant, which in turn relies on a thorough understanding of the patient's knee joint anatomy and alignment.

Coronal Plane Alignment of the Knee (CPAK) is a classification system for knee alignment that may provide insights into the balance and utility of alignment paradigms in TKA. Prior systems have utilized the CPAK classifier for two-dimensional (2D) knee analysis from frontal full leg standing x-rays. From the full leg x-ray, bony landmarks are used to identify the femur mechanical axis, tibia mechanical axis, joint line of the distal femur, and joint line of the proximal tibia. These axes are used to identify two primary angles within the plane of the x-ray: the lateral distal femoral angle (LDFA) and the medial proximal tibial angle (MPTA). These angles are then combined to determine two alignment metrics: the arithmetic hip-knee-ankle angle (aHKA), and the joint line obliquity (JLO).

In the field of TKAs, there is a recognized demand for improved surgical planning methods that can enhance patient outcomes. Specifically, there is a pressing demand for a method that can leverage the benefits of surgical robotic systems which may perform a modified three-dimensional Coronal Plane Alignment of the Knee analysis intraoperatively. This would allow for real-time, data-driven decision making regarding implant selection and positioning, thereby potentially reducing patient pain, improving post-operative function, and decreasing surgical time.

### SUMMARY

In some embodiments, a method for planning and executing a robotic-assisted total knee arthroplasty includes receiving, by a processor, surface data corresponding to boney anatomy of a knee; generating, by the processor, a three-dimensional model based on the surface data; generating, by the processor, three-dimensional Coronal Plane Alignment of the Knee (CPAK) classification data based on the three-dimensional model; and generating, by the processor, an optimal implant selection and implant position based on the three-dimensional CPAK classification data.

In some embodiments, the method includes robotically assisting, by the processor, the total knee arthroplasty based on the optimal implant selection and implant position.

In some embodiments, receiving the surface data includes receiving the surface data from a tracking system tracking a point probe.

In some embodiments, the three-dimensional model is further based off of an atlas model.

In some embodiments, generating the optimal implant selection and implant position further includes utilizing, by the processor, a machine learning algorithm to cluster the three-dimensional CPAK classification data according to a preoperative deformity.

In some embodiments, the machine learning algorithm is further configured to cluster the three-dimensional CPAK classification data according to surgeon preference.

In some embodiment, the method includes analyzing, by the processor, the three-dimensional model utilizing a biomechanics simulation, wherein the three-dimensional CPAK classification data is further based on the biomechanics simulation.

In some embodiments, the surface data is received intra-operatively.

In some embodiments, the three-dimensional CPAK classification data includes data representing the knee in a plurality of flexion angles.

In some embodiments, the method includes generating, by the processor, a customized patient-specific instrumentation guide based on the three-dimensional CPAK classification data.

In some embodiments, a system for planning and executing a robotic-assisted total knee arthroplasty includes a processor. The processor may be configured to receive surface data corresponding to boney anatomy of a knee; generate a three-dimensional model based on the surface data; generate three-dimensional Coronal Plane Alignment of the Knee (CPAK) classification data based on the three-dimensional model; and generate an optimal implant selection and implant position based on the three-dimensional CPAK classification data.

In some embodiments, the processor is further configured to robotically assist the total knee arthroplasty based on the optimal implant selection and implant position.

In some embodiments, the processor is further configured to receive the surface data from a tracking system tracking a point probe.

In some embodiments, the processor is further configured to utilize a machine learning algorithm to cluster the three-dimensional CPAK classification data according to a preoperative deformity.

In some embodiments, the machine learning algorithm is further configured to cluster the three-dimensional CPAK classification data according to surgeon preference.

In some embodiments, the processor is further configured to analyze the three-dimensional model utilizing a biomechanics simulation, wherein the three-dimensional CPAK classification data is further based on the biomechanics simulation.

In some embodiments, the surface data is received intra-operatively.

In some embodiments, the three-dimensional CPAK classification data includes data representing the knee in a plurality of flexion angles.

In some embodiments, the processor is further configured to generate a customized patient-specific instrumentation guide based on the three-dimensional CPAK classification data.

In some embodiments, a computer-readable medium having stored thereon instructions that, when executed by a processor, cause the processor to perform operations including receiving, by a processor, surface data corresponding to boney anatomy of a knee; generating, by the processor, a three-dimensional model based on the surface data; generating, by the processor, three-dimensional Coronal Plane Alignment of the Knee (CPAK) classification data based on the three-dimensional model; and generating, by the processor, an optimal implant selection and implant position based on the three-dimensional CPAK classification data.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts an operating theatre including an illustrative computer-assisted surgical system (CASS) in accordance with an embodiment.

FIG. 2A depicts illustrative control instructions that a surgical computer provides to other components of a CASS in accordance with an embodiment.

FIG. 2B depicts illustrative control instructions that components of a CASS provide to a surgical computer in accordance with an embodiment.

FIG. 2C depicts an illustrative implementation in which a surgical computer is connected to a surgical device in accordance with an embodiment.

FIG. 3 depicts an illustrative CPAK classification system for stratifying knees into phenotype groups in accordance with an embodiment.

FIG. 4 illustrates an illustrative method for surgical planning and implant selection using CPAK classification data in accordance with an embodiment.

FIG. 5 presents an illustrative graphical user interface for personalized planning in accordance with an embodiment.

FIG. 6 illustrates the capture of metrics on an orthogonal view of a knee joint before and after surgical implant positioning in accordance with an embodiment.

FIG. 7 presents orthogonal views of the upper portions of a femur and a tibia, illustrating the pre-operative joint lines and angles used in CPAK classification in accordance with an embodiment.

FIG. 8 shows an illustrative AI clustering algorithm for surgical planning in accordance with an embodiment.

FIG. 9 illustrates a block diagram of an exemplary data processing system in which embodiments are implemented.

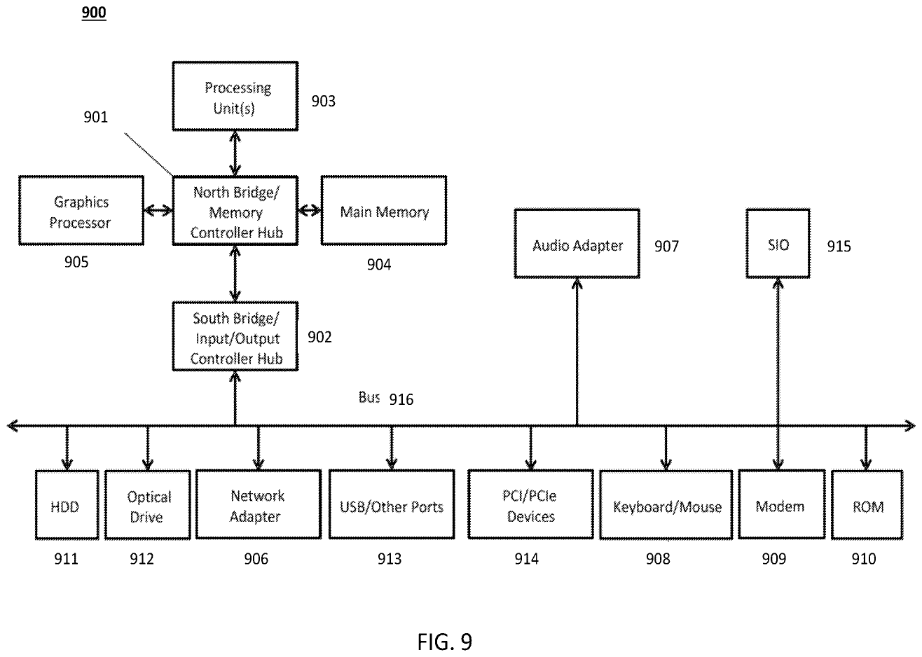

### DETAILED DESCRIPTION

This disclosure is not limited to the particular systems, devices and methods described, as these may vary. The terminology used in the description is for the purpose of describing the particular versions or embodiments only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

### Definitions

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patients worn or damaged acetabulum. While the term “implant” is generally considered to denote a man-made structure (as contrasted with a transplant), for the purposes of this specification an implant can include a biological tissue or material transplanted to replace or enhance a biological structure.

For the purposes of this disclosure, the term “native” in this context refers to the original, natural anatomy of the patient before any surgical alterations.

For the purposes of this disclosure, the term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

For the purposes of this disclosure, the terms “distract,” “distracting,” or “distraction” are used to refer to displacement of a first point with respect to a second point. For example, the first point and the second point may correspond to surfaces of a joint. In some embodiments herein, a joint may be distracted, i.e., portions of the joint may be separated and/or moved with respect to one another to place the joint under tension. In some embodiments, a first portion of the joint be a surface of a scapula and a second portion of the joint may be a surface of a humerus such that separation occurs between the bones of the joint. In additional embodiments, a first portion of the joint may be a first portion of a humeral implant component or a humeral trial implant and a second portion of the joint may be a second portion of the humeral implant component or the humeral trial implant that is movable with respect to the first portion (e.g., a humeral component and a spacer). Accordingly, separation may occur between the portions of the humeral implant component or the humeral trial implant (i.e., intra-implant separation). Throughout the disclosure herein, the described embodiments may be collectively referred to as distraction of the joint.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly demarcated. For example, a reference to a surgeon also could apply, in some embodiments to a technician or nurse.

The systems, methods, and devices disclosed herein are particularly well adapted for surgical procedures that utilize surgical navigation systems, such as the CORI® surgical navigation system. CORI is a registered trademark of SMITH & NEPHEW, INC. of Memphis, TN.

### Cass Ecosystem Overview

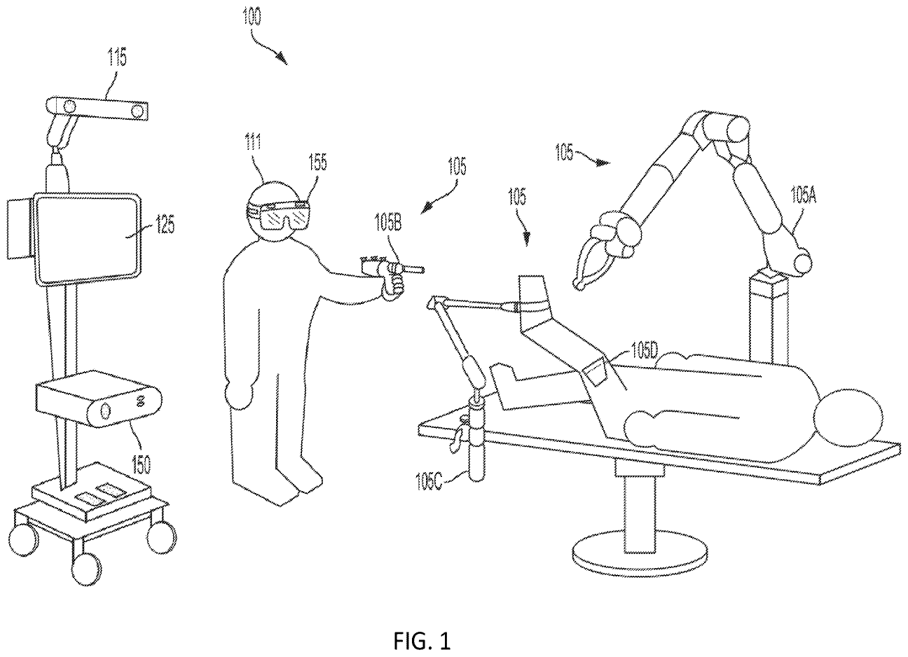

FIG. 1 provides an illustration of an example computer-assisted surgical system (CASS) 100, according to some embodiments. As described in further detail in the sections that follow, the CASS uses computers, robotics, and imaging technology to aid surgeons in performing orthopedic surgery procedures such as total knee arthroplasty (TKA), unicondylar knee arthroplasty (UKA), or total hip arthroplasty (THA). For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An Effector Platform 105 positions surgical tools relative to a patient during surgery. The exact components of the Effector Platform 105 will vary, depending on the embodiment employed. For example, for a knee surgery, the Effector Platform 105 may include an End Effector 105B that holds surgical tools or instruments during their use. The End Effector 105B may be a handheld device or instrument used by the surgeon (e.g., a CORI® hand piece or a cutting guide or jig) or, alternatively, the End Effector 105B can include a device or instrument held or positioned by a robotic arm 105A. While one robotic arm 105A is illustrated in FIG. 1, in some embodiments there may be multiple devices. As examples, there may be one robotic arm 105A on each side of an operating table T or two devices on one side of the table T. The robotic arm 105A may be mounted directly to the table T, be located next to the table T on a floor platform (not shown), mounted on a floor-to-ceiling pole, or mounted on a wall or ceiling of an operating room. The floor platform may be fixed or moveable. In one particular embodiment, the robotic arm 105A is mounted on a floor-to-ceiling pole located between the patient's legs or feet. In some embodiments, the End Effector 105B may include a suture holder or a stapler to assist in closing wounds. Further, in the case of two robotic arms 105A, the surgical computer 150 can drive the robotic arms 105A to work together to suture the wound at closure. Alternatively, the surgical computer 150 can drive one or more robotic arms 105A to staple the wound at closure.

The Effector Platform 105 can include a Limb Positioner 105C for positioning the patient's limbs during surgery. One example of a Limb Positioner 105C is the SMITH AND NEPHEW SPIDER2 system. The Limb Positioner 105C may be operated manually by the surgeon or alternatively change limb positions based on instructions received from the Surgical Computer 150 (described below). While one Limb Positioner 105C is illustrated in FIG. 1, in some embodiments there may be multiple devices. As examples, there may be one Limb Positioner 105C on each side of the operating table T or two devices on one side of the table T. The Limb Positioner 105C may be mounted directly to the table T, be located next to the table T on a floor platform (not shown), mounted on a pole, or mounted on a wall or ceiling of an operating room. In some embodiments, the Limb Positioner 105C can be used in non-conventional ways, such as a retractor or specific bone holder. The Limb Positioner 105C may include, as examples, an ankle boot, a soft tissue clamp, a bone clamp, or a soft-tissue retractor spoon, such as a hooked, curved, or angled blade. In some embodiments, the Limb Positioner 105C may include a suture holder to assist in closing wounds.

The Effector Platform 105 may include tools, such as a screwdriver, light or laser, to indicate an axis or plane, bubble level, pin driver, pin puller, plane checker, pointer, finger, or some combination thereof.

Resection Equipment 110 (not shown in FIG. 1) performs bone or tissue resection using, for example, mechanical, ultrasonic, or laser techniques. Examples of Resection Equipment 110 include drilling devices, burring devices, oscillatory sawing devices, vibratory impaction devices, reamers, ultrasonic bone cutting devices, radio frequency ablation devices, reciprocating devices (such as a rasp or broach), and laser ablation systems. In some embodiments, the Resection Equipment 110 is held and operated by the surgeon during surgery. In other embodiments, the Effector Platform 105 may be used to hold the Resection Equipment 110 during use.

The Effector Platform 105 also can include a cutting guide or jig 105D that is used to guide saws or drills used to resect tissue during surgery. Such cutting guides 105D can be formed integrally as part of the Effector Platform 105 or robotic arm 105A or cutting guides can be separate structures that can be matingly and/or removably attached to the Effector Platform 105 or robotic arm 105A. The Effector Platform 105 or robotic arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The Tracking System 115 uses one or more sensors to collect real-time position data that locates the patient's anatomy and surgical instruments. For example, for TKA procedures, the Tracking System may provide a location and orientation of the End Effector 105B during the procedure. In addition to positional data, data from the Tracking System 115 also can be used to infer velocity/acceleration of anatomy/instrumentation, which can be used for tool control. In some embodiments, the Tracking System 115 may use a tracker array attached to the End Effector 105B to determine the location and orientation of the End Effector 105B. The position of the End Effector 105B may be inferred based on the position and orientation of the Tracking System 115 and a known relationship in three-dimensional space between the Tracking System 115 and the End Effector 105B. Various types of tracking systems may be used in various embodiments of the present invention including, without limitation, Infrared (IR) tracking systems, electromagnetic (EM) tracking systems, video or image based tracking systems, and ultrasound registration and tracking systems. Using the data provided by the tracking system 115, the surgical computer 150 can detect objects and prevent collision. For example, the surgical computer 150 can prevent the robotic arm 105A and/or the End Effector 105B from colliding with soft tissue.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some embodiments, this can include stereoscopic, tri-scopic, quad-scopic, etc. imaging. In addition to the camera array, which in some embodiments is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system. In some embodiments, the camera may be mounted on the robotic arm 105A.

In some embodiments, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some embodiments, certain markers, such as fiducial marks that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one-or two-dimensional optical codes (barcode, QR code, etc.) can be affixed to objects in the theater to provide passive identification that can occur based on image analysis. If these codes are placed asymmetrically on an object, they also can be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, in some embodiments, augmented reality (AR) headsets can be worn by surgeons and other staff to provide additional camera angles and tracking capabilities. In this case, the infrared/time of flight sensor data, which is predominantly used for hand/gesture detection, can build correspondence between the AR headset and the tracking system of the robotic system using sensor fusion techniques. This can be used to calculate a calibration matrix that relates the optical camera coordinate frame to the fixed holographic world frame.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient also can involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process also can include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

A Tissue Navigation System 120 (not shown in FIG. 1) provides the surgeon with intraoperative, real-time visualization for the patient's bone, cartilage, muscle, nervous, and/or vascular tissues surrounding the surgical area. Examples of systems that may be employed for tissue navigation include fluorescent imaging systems and ultrasound systems.

The Display 125 provides graphical user interfaces (GUIs) that display images collected by the Tissue Navigation System 120 as well other information relevant to the surgery. For example, in one embodiment, the Display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The Display 125 may include, for example, one or more computer monitors. As an alternative or supplement to the Display 125, one or more members of the surgical staff may wear an Augmented Reality (AR) Head Mounted Device (HMD). For example, in FIG. 1 the Surgeon 111 is wearing an AR HMD 155 that may, for example, overlay pre-operative image data on the patient or provide surgical planning suggestions. In one embodiment, a tracker array-mounted surgical tool could be detected by both the IR camera and an AR headset (HMD) using sensor fusion techniques without the need for any “intermediate” calibration rigs. This near-depth, time-of-flight sensing camera located in the HMD could be used for hand/gesture detection. The headset's sensor API can be used to expose IR and depth image data and carryout image processing using, for example, C++ with OpenCV. This approach allows the relationship between the CASS and the virtual coordinate frame to be determined and the headset sensor data (i.e., IR in combination with depth images) to isolate the CASS tracker arrays. The image processing system on the HMD can locate the surgical tool in a fixed holographic world frame and the CASS IR camera can locate the surgical tool relative to its camera coordinate frame. This relationship can be used to calculate a calibration matrix that relates the CASS IR camera coordinate frame to the fixed holographic world frame. This means that if a calibration matrix has previously been calculated, the surgical tool no longer needs to be visible to the AR headset. However, a recalculation may be necessary if the CASS camera is accidentally moved in the workflow. Various example uses of the AR HMD 155 in surgical procedures are detailed in the sections that follow.

Surgical Computer 150 provides control instructions to various components of the CASS 100, collects data from those components, and provides general processing for various data needed during surgery. In some embodiments, the Surgical Computer 150 is a general-purpose computer. In other embodiments, the Surgical Computer 150 may be a parallel computing platform that uses multiple central processing units (CPUs) or graphics processing units (GPU) to perform processing. In some embodiments, the Surgical Computer 150 is connected to a remote server over one or more computer networks (e.g., the Internet). The remote server can be used, for example, for storage of data or execution of computationally intensive processing tasks.

Various techniques generally known in the art can be used for connecting the Surgical Computer 150 to the other components of the CASS 100. Moreover, the computers can connect to the Surgical Computer 150 using a mix of technologies. For example, the End Effector 105B may connect to the Surgical Computer 150 over a wired (i.e., serial) connection. The Tracking System 115, Tissue Navigation System 120, and Display 125 can similarly be connected to the Surgical Computer 150 using wired connections. Alternatively, the Tracking System 115, Tissue Navigation System 120, and Display 125 may connect to the Surgical Computer 150 using wireless technologies such as, without limitation, Wi-Fi, Bluetooth, Near Field Communication (NFC), or ZigBee.

### Robotic Arm

In some embodiments, the CASS 100 includes a robotic arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105A may have multiple degrees of freedom (like a Spider device) and have the ability to be locked in place (e.g., by a press of a button, voice activation, a surgeon removing a hand from the robotic arm, or other method).

In some embodiments, movement of the robotic arm 105A may be effectuated by use of a control panel built into the robotic arm system. For example, a display screen may include one or more input sources, such as physical buttons or a user interface having one or more icons, that direct movement of the robotic arm 105A. The surgeon or other healthcare professional may engage with the one or more input sources to position the robotic arm 105A when performing a surgical procedure.

A tool or an end effector 105B attached or integrated into a robotic arm 105A may include, without limitation, a burring device, a scalpel, a cutting device, a retractor, a joint tensioning device, or the like. In embodiments in which an end effector 105B is used, the end effector may be positioned at the end of the robotic arm 105A such that any motor control operations are performed within the robotic arm system. In embodiments in which a tool is used, the tool may be secured at a distal end of the robotic arm 105A, but motor control operation may reside within the tool itself.

The robotic arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105A, the robotic arm may provide some resistance to prevent the robotic arm from moving too fast or having too many degrees of freedom active at once. The position and the lock status of the robotic arm 105A may be tracked, for example, by a controller or the Surgical Computer 150.

In some embodiments, the robotic arm 105A can be moved by hand (e.g., by the surgeon) or with internal motors into its ideal position and orientation for the task being performed. In some embodiments, the robotic arm 105A may be enabled to operate in a “free” mode that allows the surgeon to position the arm into a desired position without being restricted. While in the free mode, the position and orientation of the robotic arm 105A may still be tracked as described above. In one embodiment, certain degrees of freedom can be selectively released upon input from user (e.g., surgeon) during specified portions of the surgical plan tracked by the Surgical Computer 150. Designs in which a robotic arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A robotic arm 105A or end effector 105B can include a trigger or other means to control the power of a saw or drill. Engagement of the trigger or other means by the surgeon can cause the robotic arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the robotic arm 105A or end effector 105B in an automatic mode that brings the robotic arm or end effector into the proper position with respect to the patient's anatomy in order to perform the necessary resections. The CASS 100 also can place the robotic arm 105A or end effector 105B in a collaborative mode that allows the surgeon to manually manipulate and position the robotic arm or end effector into a particular location. The collaborative mode can be configured to allow the surgeon to move the robotic arm 105A or end effector 105B medially or laterally, while restricting movement in other directions. As discussed, the robotic arm 105A or end effector 105B can include a cutting device (saw, drill, and burr) or a cutting guide or jig 105D that will guide a cutting device. In other embodiments, movement of the robotic arm 105A or robotically controlled end effector 105B can be controlled entirely by the CASS 100 without any, or with only minimal, assistance or input from a surgeon or other medical professional. In still other embodiments, the movement of the robotic arm 105A or robotically controlled end effector 105B can be controlled remotely by a surgeon or other medical professional using a control mechanism separate from the robotic arm or robotically controlled end effector device, for example using a joystick or interactive monitor or display control device.

A robotic arm 105A may be used for holding the retractor. For example, in one embodiment, the robotic arm 105A may be moved into the desired position by the surgeon. At that point, the robotic arm 105A may lock into place. In some embodiments, the robotic arm 105A is provided with data regarding the patient's position, such that if the patient moves, the robotic arm can adjust the retractor position accordingly. In some embodiments, multiple robotic arms may be used, thereby allowing multiple retractors to be held or for more than one activity to be performed simultaneously (e.g., retractor holding & reaming).

The robotic arm 105A may also be used to help stabilize the surgeon's hand while making a femoral neck cut. In this application, control of the robotic arm 105A may impose certain restrictions to prevent soft tissue damage from occurring. For example, in one embodiment, the Surgical Computer 150 tracks the position of the robotic arm 105A as it operates. If the tracked location approaches an area where tissue damage is predicted, a command may be sent to the robotic arm 105A causing it to stop. Alternatively, where the robotic arm 105A is automatically controlled by the Surgical Computer 150, the Surgical Computer may ensure that the robotic arm is not provided with any instructions that cause it to enter areas where soft tissue damage is likely to occur. The Surgical Computer 150 may impose certain restrictions on the surgeon to prevent the surgeon from reaming too far into the medial wall of the acetabulum or reaming at an incorrect angle or orientation.

In some embodiments, the robotic arm 105A may be used to hold a cup impactor at a desired angle or orientation during cup impaction. When the final position has been achieved, the robotic arm 105A may prevent any further seating to prevent damage to the pelvis.

The surgeon may use the robotic arm 105A to position the broach handle at the desired position and allow the surgeon to impact the broach into the femoral canal at the desired orientation. In some embodiments, once the Surgical Computer 150 receives feedback that the broach is fully seated, the robotic arm 105A may restrict the handle to prevent further advancement of the broach.

The robotic arm 105A may also be used for resurfacing applications. For example, the robotic arm 105A may stabilize the surgeon while using traditional instrumentation and provide certain restrictions or limitations to allow for proper placement of implant components (e.g., guide wire placement, chamfer cutter, sleeve cutter, plan cutter, etc.). Where only a burr is employed, the robotic arm 105A may stabilize the surgeon's handpiece and may impose restrictions on the handpiece to prevent the surgeon from removing unintended bone in contravention of the surgical plan.

The robotic arm 105A may be a passive arm. As an example, the robotic arm 105A may be a CIRQ robot arm available from Brainlab AG. CIRQ is a registered trademark of Brainlab AG, Olof-Palme-Str. 9 81829, München, FED REP of GERMANY. In one particular embodiment, the robotic arm 105A is an intelligent holding arm as disclosed in U.S. patent application Ser. No. 15/525,585 to Krinninger et al., U.S. patent application Ser. No. 15/561,042 to Nowatschin et al., U.S. patent application Ser. No. 15/561,048 to Nowatschin et al., and U.S. Pat. No. 10,342,636 to Nowatschin et al., the entire contents of each of which is herein incorporated by reference.

### Surgical Procedure Data Generation and Collection

The various services that are provided by medical professionals to treat a clinical condition are collectively referred to as an “episode of care.” For a particular surgical intervention, the episode of care can include three phases: pre-operative, intra-operative, and post-operative. During each phase, data is collected or generated that can be used to analyze the episode of care in order to understand various features of the procedure and identify patterns that may be used, for example, in training models to make decisions with minimal human intervention. The data collected over the episode of care may be stored at the Surgical Computer 150 or the Surgical Data Server 180 as a complete dataset. Thus, for each episode of care, a dataset exists that comprises all of the data collectively pre-operatively about the patient, all of the data collected or stored by the CASS 100 intra-operatively, and any post-operative data provided by the patient or by a healthcare professional monitoring the patient.

As explained in further detail, the data collected during the episode of care may be used to enhance performance of the surgical procedure or to provide a holistic understanding of the surgical procedure and the patient outcomes. For example, in some embodiments, the data collected over the episode of care may be used to generate a surgical plan. In one embodiment, a high-level, pre-operative plan is refined intra-operatively as data is collected during surgery. In this way, the surgical plan can be viewed as dynamically changing in real-time or near real-time as new data is collected by the components of the CASS 100. In other embodiments, pre-operative images or other input data may be used to develop a robust plan preoperatively that is simply executed during surgery. In this case, the data collected by the CASS 100 during surgery may be used to make recommendations that ensure that the surgeon stays within the pre-operative surgical plan. For example, if the surgeon is unsure how to achieve a certain prescribed cut or implant alignment, the Surgical Computer 150 can be queried for a recommendation. In still other embodiments, the pre-operative and intra-operative planning approaches can be combined such that a robust pre-operative plan can be dynamically modified, as necessary or desired, during the surgical procedure. In some embodiments, a biomechanics-based model of patient anatomy contributes simulation data to be considered by the CASS 100 in developing preoperative, intraoperative, and post-operative/rehabilitation procedures to optimize implant performance outcomes for the patient.

Aside from changing the surgical procedure itself, the data gathered during the episode of care may be used as an input to other procedures ancillary to the surgery. For example, in some embodiments, implants can be designed using episode of care data. Example data-driven techniques for designing, sizing, and fitting implants are described in U.S. Pat. No. 10,064,686, filed Aug. 15, 2011, and entitled “Systems and Methods for Optimizing Parameters for Orthopaedic Procedures”; U.S. Pat. No. 10,102,309, filed Jul. 20, 2012 and entitled “Systems and Methods for Optimizing Fit of an Implant to Anatomy”; and U.S. Pat. No. 8,078,440, filed Sep. 19, 2008 and entitled “Operatively Tuning Implants for Increased Performance,” the entire contents of each of which are hereby incorporated by reference into this patent application.

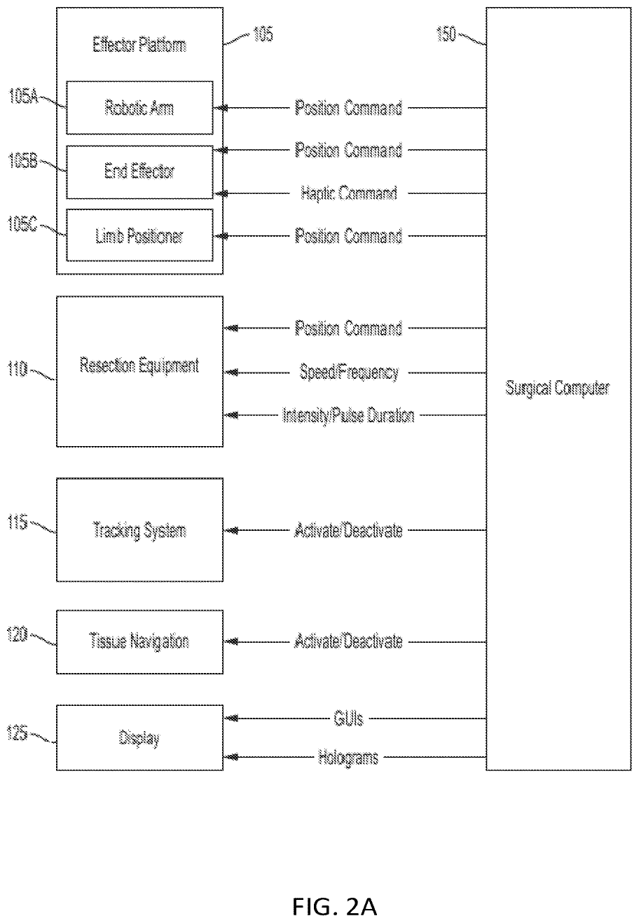

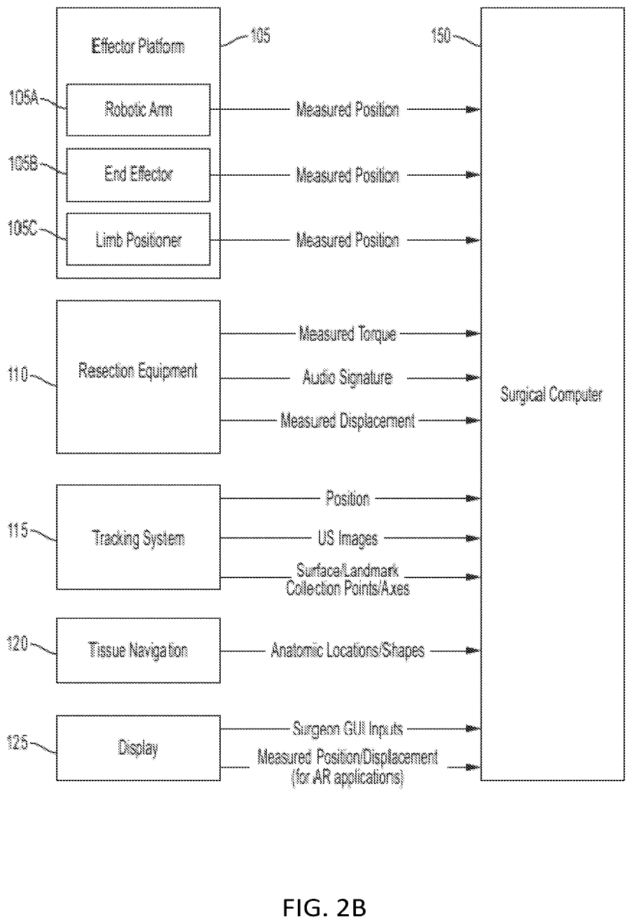

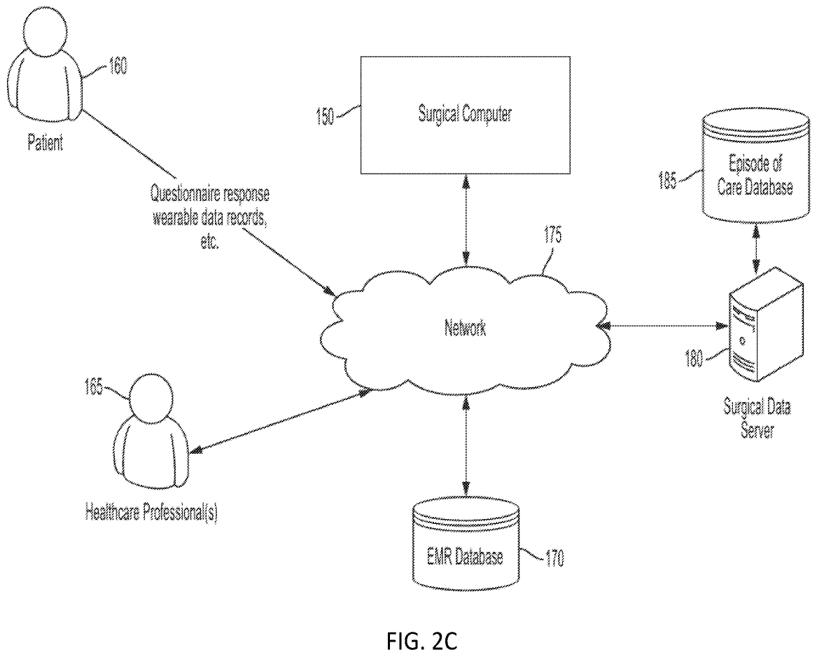

Furthermore, the data can be used for educational, training, or research purposes. For example, using the network-based approach described below in FIG. 2C, other doctors or students can remotely view surgeries in interfaces that allow them to selectively view data as it is collected from the various components of the CASS 100. After the surgical procedure, similar interfaces may be used to “playback” a surgery for training or other educational purposes, or to identify the source of any issues or complications with the procedure.

Data acquired during the pre-operative phase generally includes all information collected or generated prior to the surgery. Thus, for example, information about the patient may be acquired from a patient intake form or electronic medical record (EMR). Examples of patient information that may be collected include, without limitation, patient demographics, diagnoses, medical histories, progress notes, vital signs, medical history information, allergies, and lab results. The pre-operative data may also include images related to the anatomical area of interest. These images may be captured, for example, using Magnetic Resonance Imaging (MRI), Computed Tomography (CT), X-ray, ultrasound, or any other modality known in the art. The pre-operative data may also comprise quality of life data captured from the patient. For example, in one embodiment, pre-surgery patients use a mobile application (“app”) to answer questionnaires regarding their current quality of life. In some embodiments, preoperative data used by the CASS 100 includes demographic, anthropometric, cultural, or other specific traits about a patient that can coincide with activity levels and specific patient activities to customize the surgical plan to the patient. For example, certain cultures or demographics may be more likely to use a toilet that requires squatting on a daily basis.

FIGS. 2A and 2B provide examples of data that may be acquired during the intra-operative phase of an episode of care. These examples are based on the various components of the CASS 100 described above with reference to FIG. 1; however, it should be understood that other types of data may be used based on the types of equipment used during surgery and their use.

FIG. 2A shows examples of some of the control instructions that the Surgical Computer 150 provides to other components of the CASS 100, according to some embodiments. Note that the example of FIG. 2A assumes that the components of the Effector Platform 105 are each controlled directly by the Surgical Computer 150. In embodiments where a component is manually controlled by the Surgeon 111, instructions may be provided on the Display 125 or AR HMD 155 instructing the Surgeon 111 how to move the component.

The various components included in the Effector Platform 105 are controlled by the Surgical Computer 150 providing position commands that instruct the component where to move within a coordinate system. In some embodiments, the Surgical Computer 150 provides the Effector Platform 105 with instructions defining how to react when a component of the Effector Platform 105 deviates from a surgical plan. These commands are referenced in FIG. 2A as “haptic” commands. For example, the End Effector 105B may provide a force to resist movement outside of an area where resection is planned. Other commands that may be used by the Effector Platform 105 include vibration and audio cues.

In some embodiments, the end effectors 105B of the robotic arm 105A are operatively coupled with cutting guide 105D. In response to an anatomical model of the surgical scene, the robotic arm 105A can move the end effectors 105B and the cutting guide 105D into position to match the location of the femoral or tibial cut to be performed in accordance with the surgical plan. This can reduce the likelihood of error, allowing the vision system and a processor utilizing that vision system to implement the surgical plan to place a cutting guide 105D at the precise location and orientation relative to the tibia or femur to align a cutting slot of the cutting guide with the cut to be performed according to the surgical plan. Then, a surgeon can use any suitable tool, such as an oscillating or rotating saw or drill to perform the cut (or drill a hole) with perfect placement and orientation because the tool is mechanically limited by the features of the cutting guide 105D. In some embodiments, the cutting guide 105D may include one or more pin holes that are used by a surgeon to drill and screw or pin the cutting guide into place before performing a resection of the patient tissue using the cutting guide. This can free the robotic arm 105A or ensure that the cutting guide 105D is fully affixed without moving relative to the bone to be resected. For example, this procedure can be used to make the first distal cut of the femur during a total knee arthroplasty. In some embodiments, where the arthroplasty is a hip arthroplasty, cutting guide 105D can be fixed to the femoral head or the acetabulum for the respective hip arthroplasty resection. It should be understood that any arthroplasty that utilizes precise cuts can use the robotic arm 105A and/or cutting guide 105D in this manner.

The Resection Equipment 110 is provided with a variety of commands to perform bone or tissue operations. As with the Effector Platform 105, position information may be provided to the Resection Equipment 110 to specify where it should be located when performing resection. Other commands provided to the Resection Equipment 110 may be dependent on the type of resection equipment. For example, for a mechanical or ultrasonic resection tool, the commands may specify the speed and frequency of the tool. For Radiofrequency Ablation (RFA) and other laser ablation tools, the commands may specify intensity and pulse duration.

Some components of the CASS 100 do not need to be directly controlled by the Surgical Computer 150; rather, the Surgical Computer 150 only needs to activate the component, which then executes software locally specifying the manner in which to collect data and provide it to the Surgical Computer 150. In the example of FIG. 2A, there are two components that are operated in this manner: the Tracking System 115 and the Tissue Navigation System 120.

The Surgical Computer 150 provides the Display 125 with any visualization that is needed by the Surgeon 111 during surgery. For monitors, the Surgical Computer 150 may provide instructions for displaying images, GUIs, etc. using techniques known in the art. The display 125 can include various portions of the workflow of a surgical plan. During the registration process, for example, the display 125 can show a preoperatively constructed 3D bone model and depict the locations of the probe as the surgeon uses the probe to collect locations of anatomical landmarks on the patient. The display 125 can include information about the surgical target area. For example, in connection with a TKA, the display 125 can depict the mechanical and anatomical axes of the femur and tibia. The display 125 can depict varus and valgus angles for the knee joint based on a surgical plan, and the CASS 100 can depict how such angles will be affected if contemplated revisions to the surgical plan are made. Accordingly, the display 125 is an interactive interface that can dynamically update and display how changes to the surgical plan would impact the procedure and the final position and orientation of implants installed on bone.

As the workflow progresses to preparation of bone cuts or resections, the display 125 can depict the planned or recommended bone cuts before any cuts are performed. The surgeon 111 can manipulate the image display to provide different anatomical perspectives of the target area and can have the option to alter or revise the planned bone cuts based on intraoperative evaluation of the patient. The display 125 can depict how the chosen implants would be installed on the bone if the planned bone cuts are performed. If the surgeon 111 choses to change the previously planned bone cuts, the display 125 can depict how the revised bone cuts would change the position and orientation of the implant when installed on the bone.

The display 125 can provide the surgeon 111 with a variety of data and information about the patient, the planned surgical intervention, and the implants. Various patient-specific information can be displayed, including real-time data concerning the patient's health such as heart rate, blood pressure, etc. The display 125 also can include information about the anatomy of the surgical target region including the location of landmarks, the current state of the anatomy (e.g., whether any resections have been made, the depth and angles of planned and executed bone cuts), and future states of the anatomy as the surgical plan progresses. The display 125 also can provide or depict additional information about the surgical target region. For a TKA, the display 125 can provide information about the gaps (e.g., gap balancing) between the femur and tibia and how such gaps will change if the planned surgical plan is carried out. For a TKA, the display 125 can provide additional relevant information about the knee joint such as data about the joint's tension (e.g., ligament laxity) and information concerning rotation and alignment of the joint. The display 125 can depict how the planned implants' locations and positions will affect the patient as the knee joint is flexed. The display 125 can depict how the use of different implants or the use of different sizes of the same implant will affect the surgical plan and preview how such implants will be positioned on the bone. The CASS 100 can provide such information for each of the planned bone resections in a TKA or THA. In a TKA, the CASS 100 can provide robotic control for one or more of the planned bone resections. For example, the CASS 100 can provide robotic control only for the initial distal femur cut, and the surgeon 111 can manually perform other resections (anterior, posterior and chamfer cuts) using conventional means, such as a 4-in-1 cutting guide or jig 105D.

The display 125 can employ different colors to inform the surgeon of the status of the surgical plan. For example, un-resected bone can be displayed in a first color, resected bone can be displayed in a second color, and planned resections can be displayed in a third color. Implants can be superimposed onto the bone in the display 125, and implant colors can change or correspond to different types or sizes of implants.

The information and options depicted on the display 125 can vary depending on the type of surgical procedure being performed. Further, the surgeon 111 can request or select a particular surgical workflow display that matches or is consistent with his or her surgical plan preferences. For example, for a surgeon 111 who typically performs the tibial cuts before the femoral cuts in a TKA, the display 125 and associated workflow can be adapted to take this preference into account. The surgeon 111 also can preselect that certain steps be included or deleted from the standard surgical workflow display. For example, if a surgeon 111 uses resection measurements to finalize an implant plan but does not analyze ligament gap balancing when finalizing the implant plan, the surgical workflow display can be organized into modules, and the surgeon can select which modules to display and the order in which the modules are provided based on the surgeon's preferences or the circumstances of a particular surgery. Modules directed to ligament and gap balancing, for example, can include pre-and post-resection ligament/gap balancing, and the surgeon 111 can select which modules to include in their default surgical plan workflow depending on whether they perform such ligament and gap balancing before or after (or both) bone resections are performed.

For more specialized display equipment, such as AR HMDs, the Surgical Computer 150 may provide images, text, etc. using the data format supported by the equipment. For example, if the Display 125 is a holography device such as the Microsoft Hololens™ or Magic Leap One™, the Surgical Computer 150 may use the Hololens Application Program Interface (API) to send commands specifying the position and content of holograms displayed in the field of view of the Surgeon 111.

In some embodiments, one or more surgical planning models may be incorporated into the CASS 100 and used in the development of the surgical plans provided to the surgeon 111. The term “surgical planning model” refers to software that simulates the biomechanics performance of anatomy under various scenarios to determine the optimal way to perform cutting and other surgical activities. For example, for knee replacement surgeries, the surgical planning model can measure parameters for functional activities, such as deep knee bends, gait, etc., and select cut locations on the knee to optimize implant placement. One example of a surgical planning model is the LIFEMOD™ simulation software from SMITH AND NEPHEW, INC. In some embodiments, the Surgical Computer 150 includes computing architecture that allows full execution of the surgical planning model during surgery (e.g., a GPU-based parallel processing environment). In other embodiments, the Surgical Computer 150 may be connected over a network to a remote computer that allows such execution, such as a Surgical Data Server 180 (see FIG. 2C). As an alternative to full execution of the surgical planning model, in some embodiments, a set of transfer functions are derived that simplify the mathematical operations captured by the model into one or more predictor equations. Then, rather than execute the full simulation during surgery, the predictor equations are used. Further details on the use of transfer functions are described in WIPO Publication No. 2020/037308, filed Aug. 19, 2019, entitled “Patient Specific Surgical Method and System,” the entirety of which is incorporated herein by reference.

FIG. 2B shows examples of some of the types of data that can be provided to the Surgical Computer 150 from the various components of the CASS 100. In some embodiments, the components may stream data to the Surgical Computer 150 in real-time or near real-time during surgery. In other embodiments, the components may queue data and send it to the Surgical Computer 150 at set intervals (e.g., every second). Data may be communicated using any format known in the art. Thus, in some embodiments, the components all transmit data to the Surgical Computer 150 in a common format. In other embodiments, each component may use a different data format, and the Surgical Computer 150 is configured with one or more software applications that enable translation of the data.

In general, the Surgical Computer 150 may serve as the central point where CASS data is collected. The exact content of the data will vary depending on the source. For example, each component of the Effector Platform 105 provides a measured position to the Surgical Computer 150. Thus, by comparing the measured position to a position originally specified by the Surgical Computer 150 (see FIG. 2B), the Surgical Computer can identify deviations that take place during surgery.

The Resection Equipment 110 can send various types of data to the Surgical Computer 150 depending on the type of equipment used. Example data types that may be sent include the measured torque, audio signatures, and measured displacement values. Similarly, the Tracking Technology 115 can provide different types of data depending on the tracking methodology employed. Example tracking data types include position values for tracked items (e.g., anatomy, tools, etc.), ultrasound images, and surface or landmark collection points or axes. The Tissue Navigation System 120 provides the Surgical Computer 150 with anatomic locations, shapes, etc. as the system operates.

Although the Display 125 generally is used for outputting data for presentation to the user, it may also provide data to the Surgical Computer 150. For example, for embodiments where a monitor is used as part of the Display 125, the Surgeon 111 may interact with a GUI to provide inputs which are sent to the Surgical Computer 150 for further processing. For AR applications, the measured position and displacement of the HMD may be sent to the Surgical Computer 150 so that it can update the presented view as needed.

During the post-operative phase of the episode of care, various types of data can be collected to quantify the overall improvement or deterioration in the patient's condition as a result of the surgery. The data can take the form of, for example, self-reported information reported by patients via questionnaires. For example, in the context of a knee replacement surgery, functional status can be measured with an Oxford Knee Score questionnaire, and the post-operative quality of life can be measured with a EQ5D-5L questionnaire. Other examples in the context of a hip replacement surgery may include the Oxford Hip Score, Harris Hip Score, and WOMAC (Western Ontario and McMaster Universities Osteoarthritis index). Such questionnaires can be administered, for example, by a healthcare professional directly in a clinical setting or using a mobile app that allows the patient to respond to questions directly. In some embodiments, the patient may be outfitted with one or more wearable devices that collect data relevant to the surgery. For example, following a knee surgery, the patient may be outfitted with a knee brace that includes sensors that monitor knee positioning, flexibility, etc. This information can be collected and transferred to the patient's mobile device for review by the surgeon to evaluate the outcome of the surgery and address any issues. In some embodiments, one or more cameras can capture and record the motion of a patient's body segments during specified activities postoperatively. This motion capture can be compared to a biomechanics model to better understand the functionality of the patient's joints and better predict progress in recovery and identify any possible revisions that may be needed.

The post-operative stage of the episode of care can continue over the entire life of a patient. For example, in some embodiments, the Surgical Computer 150 or other components comprising the CASS 100 can continue to receive and collect data relevant to a surgical procedure after the procedure has been performed. This data may include, for example, images, answers to questions, “normal” patient data (e.g., blood type, blood pressure, conditions, medications, etc.), biometric data (e.g., gait, etc.), and objective and subjective data about specific issues (e.g., knee or hip joint pain). This data may be explicitly provided to the Surgical Computer 150 or other CASS component by the patient or the patient's physician(s). Alternatively, or additionally, the Surgical Computer 150 or other CASS component can monitor the patient's EMR and retrieve relevant information as it becomes available. This longitudinal view of the patient's recovery allows the Surgical Computer 150 or other CASS component to provide a more objective analysis of the patient's outcome to measure and track success or lack of success for a given procedure. For example, a condition experienced by a patient long after the surgical procedure can be linked back to the surgery through a regression analysis of various data items collected during the episode of care. This analysis can be further enhanced by performing the analysis on groups of patients that had similar procedures and/or have similar anatomies.

In some embodiments, data is collected at a central location to provide for easier analysis and use. Data can be manually collected from various CASS components in some instances. For example, a portable storage device (e.g., USB stick) can be attached to the Surgical Computer 150 into order to retrieve data collected during surgery. The data can then be transferred, for example, via a desktop computer to the centralized storage. Alternatively, in some embodiments, the Surgical Computer 150 is connected directly to the centralized storage via a Network 175 as shown in FIG. 2C.

FIG. 2C illustrates a “cloud-based” implementation in which the Surgical Computer 150 is connected to a Surgical Data Server 180 via a Network 175. This Network 175 may be, for example, a private intranet or the Internet. In addition to the data from the Surgical Computer 150, other sources can transfer relevant data to the Surgical Data Server 180. The example of FIG. 2C shows three additional data sources: the Patient 160, Healthcare Professional(s) 165, and an EMR Database 170. Thus, the Patient 160 can send pre-operative and post-operative data to the Surgical Data Server 180, for example, using a mobile app. The Healthcare Professional(s) 165 includes the surgeon and his or her staff as well as any other professionals working with Patient 160 (e.g., a personal physician, a rehabilitation specialist, etc.). It should also be noted that the EMR Database 170 may be used for both pre-operative and post-operative data. For example, assuming that the Patient 160 has given adequate permissions, the Surgical Data Server 180 may collect the EMR of the Patient pre-surgery. Then, the Surgical Data Server 180 may continue to monitor the EMR for any updates post-surgery.

At the Surgical Data Server 180, an Episode of Care Database 185 is used to store the various data collected over a patient's episode of care. The Episode of Care Database 185 may be implemented using any technique known in the art. For example, in some embodiments, a SQL-based database may be used where all of the various data items are structured in a manner that allows them to be readily incorporated in two SQL's collection of rows and columns. However, in other embodiments a No-SQL database may be employed to allow for unstructured data, while providing the ability to rapidly process and respond to queries. As is understood in the art, the term “No-SQL” is used to define a class of data stores that are non-relational in their design. Various types of No-SQL databases may generally be grouped according to their underlying data model. These groupings may include databases that use column-based data models (e.g., Cassandra), document-based data models (e.g., MongoDB), key-value based data models (e.g., Redis), and/or graph-based data models (e.g., Allego). Any type of No-SQL database may be used to implement the various embodiments described herein and, in some embodiments, the different types of databases may support the Episode of Care Database 185.

Data can be transferred between the various data sources and the Surgical Data Server 180 using any data format and transfer technique known in the art. It should be noted that the architecture shown in FIG. 2C allows transmission from the data source to the Surgical Data Server 180, as well as retrieval of data from the Surgical Data Server 180 by the data sources. For example, as explained in detail below, in some embodiments, the Surgical Computer 150 may use data from past surgeries, machine learning models, etc. to help guide the surgical procedure.

In some embodiments, the Surgical Computer 150 or the Surgical Data Server 180 may execute a de-identification process to ensure that data stored in the Episode of Care Database 185 meets Health Insurance Portability and Accountability Act (HIPAA) standards or other requirements mandated by law. HIPAA provides a list of certain identifiers that must be removed from data during de-identification. The aforementioned de-identification process can scan for these identifiers in data that is transferred to the Episode of Care Database 185 for storage. For example, in one embodiment, the Surgical Computer 150 executes the de-identification process just prior to initiating transfer of a particular data item or set of data items to the Surgical Data Server 180. In some embodiments, a unique identifier is assigned to data from a particular episode of care to allow for re-identification of the data if necessary.

Although FIGS. 2A-C discuss data collection in the context of a single episode of care, it should be understood that the general concept can be extended to data collection from multiple episodes of care. For example, surgical data may be collected over an entire episode of care each time a surgery is performed with the CASS 100 and stored at the Surgical Computer 150 or at the Surgical Data Server 180. As explained in further detail below, a robust database of episode of care data allows the generation of optimized values, measurements, distances, or other parameters and other recommendations related to the surgical procedure. In some embodiments, the various datasets are indexed in the database or other storage medium in a manner that allows for rapid retrieval of relevant information during the surgical procedure. For example, in one embodiment, a patient-centric set of indices may be used so that data pertaining to a particular patient or a set of patients similar to a particular patient can be readily extracted. This concept can be similarly applied to surgeons, implant characteristics, CASS component versions, etc.

Further details of the management of episode of care data are described in U.S. Pat. No. 11,532,402, filed Apr. 13, 2020, and entitled “METHODS AND SYSTEMS FOR PROVIDING AN EPISODE OF CARE,” the entirety of which is incorporated herein by reference.

### Using the Point Probe to Acquire High-Resolution of Key Areas During Hip Surgeries

Use of the point probe is described in U.S. patent application Ser. No. 14/455,742 entitled “Systems and Methods for Planning and Performing Image Free Implant Revision Surgery,” the entirety of which is incorporated herein by reference. Briefly, an optically tracked point probe may be used to map the actual surface of the target bone that needs a new implant. Mapping is performed after removal of the defective or worn-out implant, as well as after removal of any diseased or otherwise unwanted bone. A plurality of points is collected on the bone surfaces by brushing or scraping the entirety of the remaining bone with the tip of the point probe. This is referred to as tracing or “painting” the bone. The collected points are used to create a three-dimensional model or surface map of the bone surfaces in the computerized planning system. The created 3D model of the remaining bone is then used as the basis for planning the procedure and necessary implant sizes. An alternative technique that uses X-rays to determine a 3D model is described in U.S. patent application Ser. No. 16/387,151, filed Apr. 17, 2019 and entitled “Three-Dimensional Selective Bone Matching” and U.S. patent application Ser. No. 16/789,430, filed Feb. 13, 2020 and entitled “Three-Dimensional Selective Bone Matching,” the entirety of each of which is incorporated herein by reference.

For hip applications, the point probe painting can be used to acquire high resolution data in key areas such as the acetabular rim and acetabular fossa. This can allow a surgeon to obtain a detailed view before beginning to ream. For example, in one embodiment, the point probe may be used to identify the floor (fossa) of the acetabulum. As is well understood in the art, in hip surgeries, it is important to ensure that the floor of the acetabulum is not compromised during reaming so as to avoid destruction of the medial wall. If the medial wall were inadvertently destroyed, the surgery would require the additional step of bone grafting. With this in mind, the information from the point probe can be used to provide operating guidelines to the acetabular reamer during surgical procedures. For example, the acetabular reamer may be configured to provide haptic feedback to the surgeon when he or she reaches the floor or otherwise deviates from the surgical plan. Alternatively, the CASS 100 may automatically stop the reamer when the floor is reached or when the reamer is within a threshold distance.

As an additional safeguard, the thickness of the area between the acetabulum and the medial wall could be estimated. For example, once the acetabular rim and acetabular fossa has been painted and registered to the pre-operative 3D model, the thickness can readily be estimated by comparing the location of the surface of the acetabulum to the location of the medial wall. Using this knowledge, the CASS 100 may provide alerts or other responses in the event that any surgical activity is predicted to protrude through the acetabular wall while reaming.

The point probe may also be used to collect high resolution data of common reference points used in orienting the 3D model to the patient. For example, for pelvic plane landmarks like the ASIS and the pubic symphysis, the surgeon may use the point probe to paint the bone to represent a true pelvic plane. Given a more complete view of these landmarks, the registration software has more information to orient the 3D model.

The point probe may also be used to collect high-resolution data describing the proximal femoral reference point that could be used to increase the accuracy of implant placement. For example, the relationship between the tip of the Greater Trochanter (GT) and the center of the femoral head is commonly used as reference point to align the femoral component during hip arthroplasty. The alignment is highly dependent on proper location of the GT; thus, in some embodiments, the point probe is used to paint the GT to provide a high-resolution view of the area. Similarly, in some embodiments, it may be useful to have a high-resolution view of the Lesser Trochanter (LT). For example, during hip arthroplasty, the Dorr Classification helps to select a stem that will maximize the ability of achieving a press-fit during surgery to prevent micromotion of femoral components post-surgery and ensure optimal bony ingrowth. As is generated understood in the art, the Dorr Classification measures the ratio between the canal width at the LT and the canal width 10 cm below the LT. The accuracy of the classification is highly dependent on the correct location of the relevant anatomy. Thus, it may be advantageous to paint the LT to provide a high-resolution view of the area.

In some embodiments, the point probe is used to paint the femoral neck to provide high-resolution data that allows the surgeon to better understand where to make the neck cut. The navigation system can then guide the surgeon as they perform the neck cut. For example, as understood in the art, the femoral neck angle is measured by placing one line down the center of the femoral shaft and a second line down the center of the femoral neck. Thus, a high-resolution view of the femoral neck (and possibly the femoral shaft as well) would provide a more accurate calculation of the femoral neck angle.

High-resolution femoral head neck data also could be used for a navigated resurfacing procedure where the software/hardware aids the surgeon in preparing the proximal femur and placing the femoral component. As is generally understood in the art, during hip resurfacing, the femoral head and neck are not removed; rather, the head is trimmed and capped with a smooth metal covering. In this case, it would be advantageous for the surgeon to paint the femoral head and cap so that an accurate assessment of their respective geometries can be understood and used to guide trimming and placement of the femoral component.

### Registration of Pre-Operative Data to Patient Anatomy Using the Point Probe

As noted above, in some embodiments, a 3D model is developed during the pre-operative stage based on 2D or 3D images of the anatomical area of interest. In such embodiments, registration between the 3D model and the surgical site is performed prior to the surgical procedure. The registered 3D model may be used to track and measure the patient's anatomy and surgical tools intraoperatively.

During the surgical procedure, landmarks are acquired to facilitate registration of this pre-operative 3D model to the patient's anatomy. For knee procedures, these points could comprise the femoral head center, distal femoral axis point, medial and lateral epicondyles, medial and lateral malleolus, proximal tibial mechanical axis point, and tibial A/P direction. For hip procedures these points could comprise the anterior superior iliac spine (ASIS), the pubic symphysis, points along the acetabular rim and within the hemisphere, the greater trochanter (GT), and the lesser trochanter (LT).

In a revision surgery, the surgeon may paint certain areas that contain anatomical defects to allow for better visualization and navigation of implant insertion. These defects can be identified based on analysis of the pre-operative images. For example, in one embodiment, each pre-operative image is compared to a library of images showing “healthy” anatomy (i.e., without defects). Any significant deviations between the patient's images and the healthy images can be flagged as a potential defect. Then, during surgery, the surgeon can be warned of the possible defect via a visual alert on the display 125 of the CASS 100. The surgeon can then paint the area to provide further detail regarding the potential defect to the Surgical Computer 150.

In some embodiments, the surgeon may use a non-contact method for registration of bony anatomy intra-incision. For example, in one embodiment, laser scanning is employed for registration. A laser stripe is projected over the anatomical area of interest and the height variations of the area are detected as changes in the line. Other non-contact optical methods, such as white light interferometry or ultrasound, may alternatively be used for surface height measurement or to register the anatomy. For example, ultrasound technology may be beneficial where there is soft tissue between the registration point and the bone being registered (e.g., ASIS, pubic symphysis in hip surgeries), thereby providing for a more accurate definition of anatomic planes.

### Three-Dimensional CPAK Characterization for Improved Robotic Outcomes

The present disclosure relates to systems and methods for improving outcomes in Total Knee Arthroplasty (TKA) procedures. More specifically, the disclosure pertains to the use of a modified three-dimensional (3D) Coronal Plane Alignment of the Knee (CPAK) analysis for pre and/or intraoperative surgical planning and postoperative evaluation. The CPAK classification, which characterizes a knee joint based on the Hip-Knee-Ankle (HKA) angle and Joint Line Obliquity metrics, can inform implant selection and positioning decisions. However, traditional CPAK classification requires a full leg X-ray, which may not be readily available or feasible in all situations.

The arithmetic hip-knee-ankle angle (aHIKA) may be calculated by subtracting the lateral distal femoral angle (LDFA) from the medial proximal tibia angle (MPTA), resulting in values centered around zero degrees for neutral knees. Negative aHKA values indicate varus leg alignments, while positive aHKA values indicate valgus leg alignments. Joint line obliquity (JLO) may be calculated by adding together the LDFA and MPTA, resulting in values centered around 180 degrees for neutral knees. JLO values less than 180 degrees indicate a joint line angled with the apex distal, while JLO values greater than 180 degrees indicate a joint line angled with the apex proximal.

In some embodiments, the present disclosure provides a method for performing a 3D CPAK analysis using automated landmarking tools to collect the requisite points from anatomical surface data of a patient's knee joint. This data may be obtained from a surgical robotic system and registered to a 3D surface for analysis. The landmarks determined on the 3D surface using kinematic modeling may then be used to generate CPAK classification data.

In some embodiments, the CPAK classification data, which may include the arithmetic hip-knee-ankle angle (aHKA) and the joint line obliquity (JLO), may be used to generate an optimized implant selection and implant position plan. This plan can be generated using Artificial Intelligence (AI) or Machine Learning (ML) algorithms, which may be trained on previously completed cases and/or surgeon preferences.

In further embodiments, the present disclosure provides a CASS 100 configured for performing the method.

In yet other embodiments, the present disclosure provides a non-transitory computer-readable medium storing instructions that, when executed by a processor, cause the processor to perform the aforementioned method.

The methods, systems, and computer-readable mediums disclosed herein may provide several advantages. For instance, they may allow for a more robust version of the 2D CPAK metric, eliminate the need for a full leg X-ray, and enable intraoperative performance of the CPAK analysis. Furthermore, they may provide a more effective way of differentiating surgical techniques and implant selection and positioning, thereby potentially leading to better postoperative outcomes such as improved kinematics, reduced patient pain, and enhanced patient function.

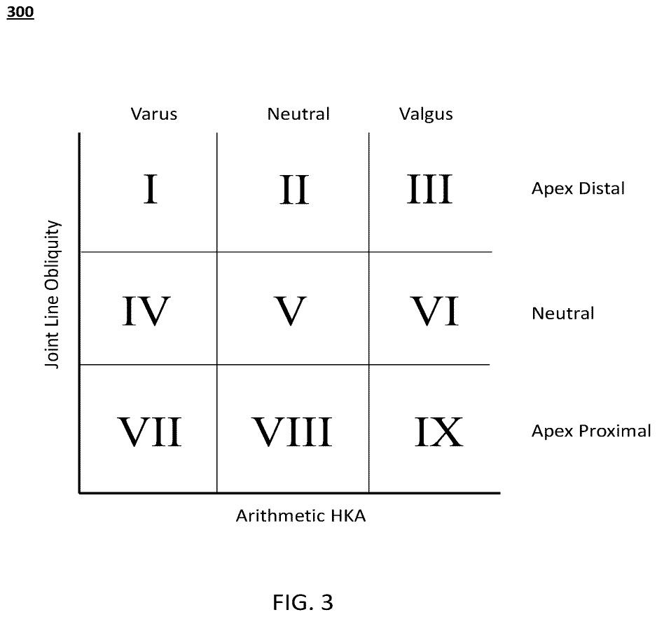

Referring to FIG. 3, an example of a CPAK classification system 300 is illustrated. The CPAK classification system 300 stratifies knees into nine phenotype groups based on the HKA angle and JLO metrics. In prior systems, these metrics are derived from bony landmarks identified on a full leg X-ray. However, according to some embodiments, a method for generating CPAK classification data based on landmarks determined on a three-dimensional (3D) surface of a patient's knee joint are provided. This method allows for a modified 3D CPAK analysis that may be performed without the prerequisite of a full leg X-ray, thereby providing a more convenient and efficient approach for planning and evaluating TKA procedures.

A CPAK classification may utilize a set of metrics, primarily the HKA angle and JLO, to categorize knee joints into distinct phenotypes. These phenotypes may assist surgeons in making informed decisions about implant selection and positioning, which are integral to the success of TKA procedures. The HKA angle provides insight into the overall alignment of the leg, indicating whether the leg is varus (angled inward) or valgus (angled outward), while the JLO offers information on the orientation of the joint line relative to the ground. By employing these metrics, the CPAK classification system may provide a more personalized approach to TKA, aiming to optimize patient outcomes by tailoring the surgical plan to the individual's knee alignment.

The current CPAK phenotype classification system stratifies knees into 9 different phenotype groups. However, any number of stratifications may be utilized. For example, the stratifications may be based on more evenly distributing the patient population, or based on patient outcomes or function, or for any other method. Furthermore, CPAK has previously only been defined to address coronal plane alignment and only while standing/at full extension. An alignment may also be considered in the sagittal or transverse planes and at various flexion angles aside from full extension.

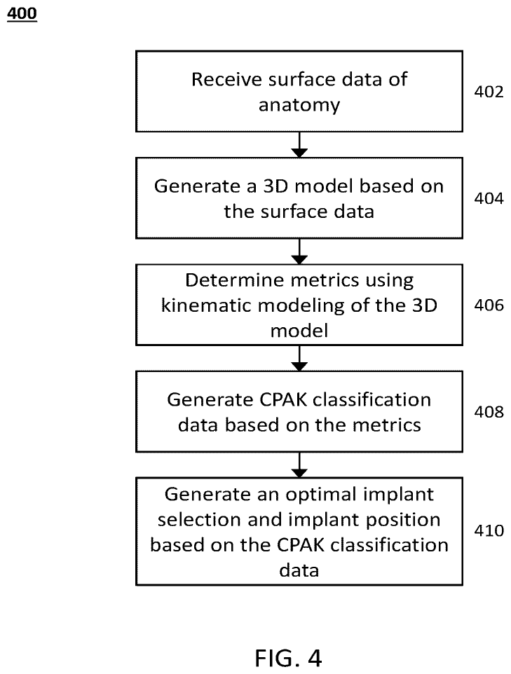

Referring now to FIG. 4, a method 400 for surgical planning and implant selection is depicted. The method 400 includes receiving 402 anatomical surface data. In some embodiments, the anatomy surface data is associated with a patient's knee (e.g., the femur and tibia). The surface data may be obtained using a point probe as disclosed herein. In further embodiments, a surgical robotic system may be equipped with imaging capabilities that allow for the capture of detailed three-dimensional anatomy surface data of the patient's knee joint.

In certain embodiments, the method 400 includes generating 404 a three-dimensional model based on the surface data. The three-dimensional model may be further based on pre-operative imagery and/or an atlas model. Generating 404 the three-dimensional model may involve various image processing techniques, including but not limited to, segmentation, alignment, and transformation processes.

In certain embodiments, metrics are determined 406 based on the 3D surface. The metrics may be determined using biomechanics simulation software (e.g., LIFEMOD). The biomechanics simulation software may determine the location of additional landmarks on the anatomy and simulate the joint at multiple flexion angles to generate metrics. The metrics may include, but are not limited to, the femoral mechanical axis, tibial mechanical axis, the joint line of the distal femur, and the joint line of the proximal tibia. The identification of these metrics can provide valuable information about the alignment and orientation of the knee joint, which can be used in additional steps of the method 400.

The method 400 may include generating 408 CPAK classification data based on the metrics. The CPAK classification data may include an aHKA and a JLO. The aHKA metric may be calculated by subtracting the LDFA from the MPTA. The JLO metric may be calculated by adding the LDFA and the MPTA together. The generation 408 of the CPAK classification data may provide a comprehensive characterization of the knee joint, which can inform the selection and positioning of the implant. In alternative embodiments, the metrics may be generated based on the three-dimensional model and/or the collected surface data.

In certain embodiments, the method 400 includes generating 410 an optimized implant selection and implant position plan based on the CPAK classification data. In some embodiments, the optimized implant selection and implant position plan is generated using an Artificial Intelligence (AI) or Machine Learning (ML) algorithm. The AI or ML algorithm may be trained on previously completed cases and/or surgeon preferences, thereby allowing for a personalized and optimized surgical plan.

In other embodiments, the method 400 may include utilizing the CPAK classification in a biomechanics-based simulation. Using the current patient CPAK value along with ideal biomechanics outcomes, a biomechanically optimized solution may be generated. This solution may inform which implant to select and where it can be positioned. This variation may be implemented in the optimized implant selection step 410 of the method 400.

In other embodiments, the method 400 may include the provision of alignment options that reflect a clinical outcome goal. These options may include “Overall balanced fit” or “Patella balance priority fit” or “Flexion stability fit”. These clinical outcome preferences each correspond to a slightly different optimized profile reflecting the priority and based upon the simulation set results. This variation may be implemented in the optimized implant selection step 410 of the method 400.

In some embodiments, the method 400 may involve the development of other characterizations that could similarly inform an optimization algorithm. For instance, patellar characterizations or femur characterizations may be used. These characterizations could provide additional insights into the patient's knee joint, which could further inform the selection and positioning of the implant. These characterizations may be generated based on the landmarks determined on the 3D model.

In other embodiments, the method 400 may involve the consideration of alignment in the sagittal or transverse planes and at various flexion angles aside from full extension. This could provide a more comprehensive understanding of the patient's knee joint alignment, which could further inform the selection and positioning of the implant. For example, landmarks may be determined not just in the coronal plane, but also in the sagittal and transverse planes and at various flexion angles. The resulting alignment data may be used to optimize implant selection and positioning.

In alternative embodiments, the method 400 may include the use of patient collected movement data for characterization. Movement data may be collected using accelerometers and/or instrumented implants. The movement data may characterize the gait of the patient. The movement data may provide additional insights into the patient's knee joint function, which can further inform the selection and positioning of the implant. In further embodiments, the movement data may be optically captured.

In some embodiments, a patient is analyzed clinically before the procedure. Patient specific movement data may be collected while the patient performs particular movement patterns (e.g., walking, climbing stairs, turning, sitting, getting into a car seat, etc.). The biomechanics motion capture analysis may utilize a biomechanics simulation suite (e.g., LIFEMOD), to quantify how the joint is moving (e.g., centers of rotation, muscle activity, function/disfunction, and/or insufficiency). The motion data may be used to classify the patient based on the type and range of rotation at each joint (e.g., full range of motion versus stiff, purely rotating versus rotating with translation, timing of knee joint rotation compared to hip, etc.), presence and type of gait imbalances or abnormality, and how that compares to the range of other pre-operative patients.

Based on the above characterization, the system may predict the presence of an abnormality in a biomechanical simulation and provide a corresponding specific surgical plans/implant positioning configured to remedy the abnormality. An AI/ML model may be utilized for characterizing the movement data and/or identifying a corresponding treatment.

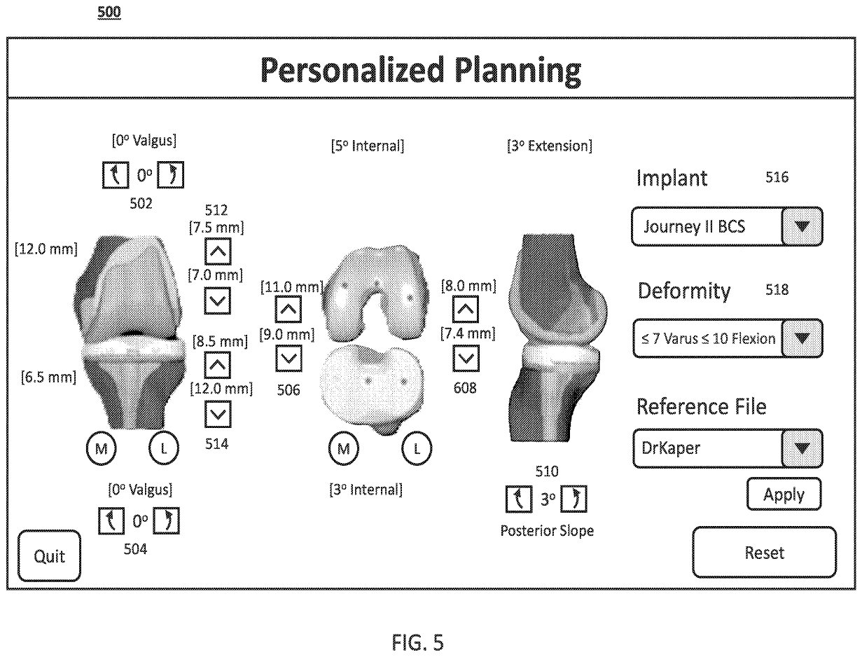

Referring now to FIG. 5, a graphical user interface (GUI) for Personalized Planning 500 is depicted. The Personalized Planning GUI 500 may allow a surgeon to adjust various parameters related to the implant selection and positioning. Modifiable parameters may vary based on coronal deformity. Resection may be based on the unaffected compartment. The interface may include controls for adjusting the femoral component varus/valgus 502 and the tibial component varus/valgus 504. These controls allow the surgeon to adjust the alignment of the femoral and tibial components in the varus/valgus direction, which can influence the overall alignment of the knee joint post-operatively.

The Personalized Planning GUI 500 may provide options for specifying the medial posterior femoral resection 506 and the lateral posterior femoral resection 508. These options may allow the surgeon to specify the amount of bone to be resected from the medial and lateral posterior aspects of the femur, which may influence the positioning of the femoral component and the overall alignment of the knee joint.

In certain embodiments, the Personalized Planning GUI 500 includes a control for adjusting the tibial component slope 510. The tibial component slope 510 may influence the flexion/extension alignment of the knee joint and may be adjusted to achieve a desired post-operative alignment.

The Personalized Planning GUI 500 may include controls for adjusting the lateral distal femoral resection 512 and the lateral tibial resection 514. These controls 512,514 may allow the surgeon to specify the amount of bone to be resected from the lateral distal femur and the lateral tibia, which may influence the positioning of the femoral and tibial components and the overall alignment of the knee joint. These controls may only be available in the presence of varus deformities.

The Personalized Planning GUI 500 may include controls for adjusting the medial distal femoral resection and the medial tibial resection. These controls may allow the surgeon to specify the amount of bone to be resected from the medial distal femur and the medial tibia, which may influence the positioning of the femoral and tibial components and the overall alignment of the knee joint. These controls may only be available in the presence of valgus deformities.

The Personalized Planning GUI 500 may include controls for implant selection 516 and deformity classification 518. The implant selection control 516 allows the surgeon to choose the appropriate implant based on the patient's specific anatomy and the desired post-operative outcome. The deformity classification control 518 enables the surgeon to classify the patient's pre-operative deformity based on specific criteria, which can inform the implant selection and positioning decisions. Example varus deformity classifications may include 0° to 7° varus and 0° to 10° flexion, 0° to 7° varus and greater than 10° flexion, greater than 7° varus and 0° to 10° flexion, and greater than 7° varus and greater than 10° flexion. Example valgus deformity classifications may include 0° to 7° valgus and 0° to 10° flexion, 0° to 7° valgus and greater than 10° flexion, greater than 7° valgus and 0° to 10° flexion, and greater than 7° valgus and greater than 10° flexion. As described herein, selecting a deformity classification may change other configurable values on the Personalized Planning GUI 500.

In some embodiments, the system may be configured to optimize one or more of these controlled values. For example, the surgeon may prompt the system to optimize all values or lock a portion thereof.

In some embodiments, the Personalized Planning GUI 500 may be implemented on a computer system or a surgical robotic system. The interface may be displayed on a monitor or a touchscreen display, allowing the surgeon to interact with the interface using a mouse, a keyboard, a stylus, or touch input. The Personalized Planning GUI 500 may provide a convenient and efficient tool for surgeons to plan and optimize TKA procedures, potentially leading to improved post-operative outcomes.

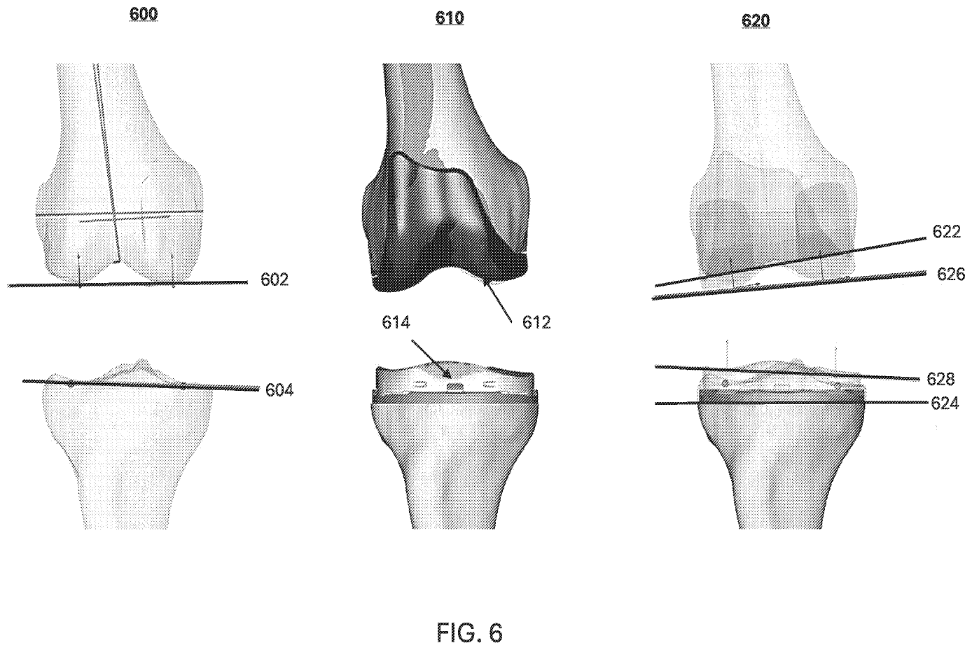

Referring now to FIG. 6, orthogonal views of a knee joint before and after surgical implant positioning are depicted. The figure illustrates the transition from the natural joint lines to the modified joint lines after the surgical placement of the implants, indicating the changes in alignment and positioning that occur during the procedure.

A pre-operative condition of the joint anatomy 600 is shown. In the pre-operative condition, landmarks may be captured to determine the femoral pre-operative joint line 602 and the tibial pre-operative joint line 604. These joint lines represent the natural alignment of the knee joint before the surgical procedure. These joint lines serve as references for the surgical planning and implant positioning process.

Pre- or intra-operatively, implant positioning 610 of the femoral implant 612 and the tibial implant 614 may occur. The femoral implant 612 is positioned on the distal end of the femur, while the tibial implant 614 is positioned on the proximal end of the tibia. The positioning of these implants may be determined based on the CPAK classification data and the optimized implant selection and implant position plan generated by the AI or ML algorithm.

A post-operative condition of the joint anatomy 620 is shown. The femoral resection plane 622 and the tibial resection plane 624 are visualized on the joint anatomy 620. The femoral resection plane 622 and the tibial resection plane 624 represent the areas of the femur and tibia, respectively, that have been resected during the surgical procedure to accommodate the implants. Additionally, the femoral post-operative joint line 626 and the tibial post-operative joint line 628 are shown. The femoral post-operative joint line 626 and the tibial post-operative joint line 628 represent the new alignment of the knee joint after the surgical placement of the implants. These reference lines may be utilized to generate useful metrics, such as CPAK classification.

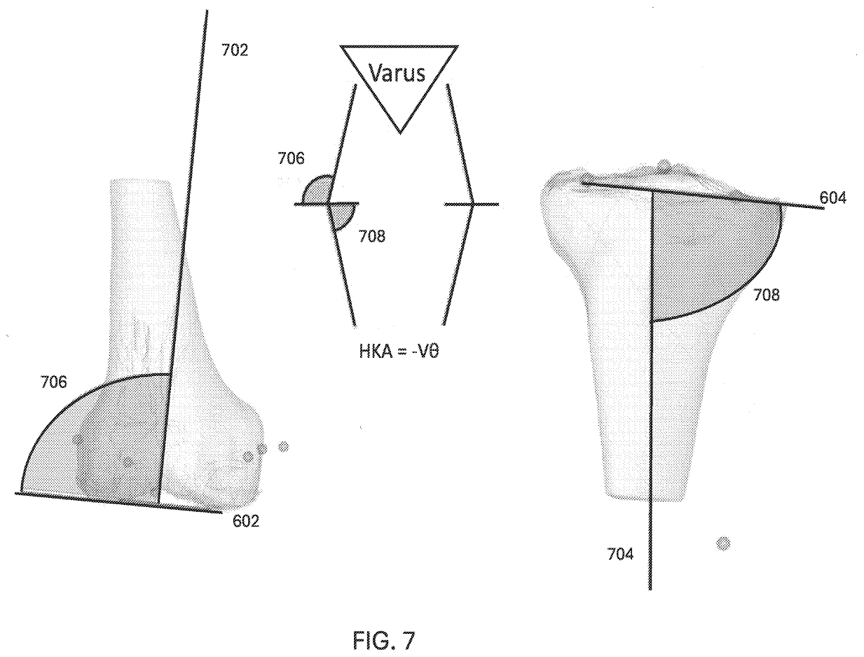

Referring now to FIG. 7, orthogonal views of the upper portions of a femur bone upper portion 700 and a tibia bone upper portion 710 are depicted, illustrating the metrics used in CPAK classification. The lateral distal femoral angle (LDFA) 706 may be determined by taking the angle between a line along the femoral normal axis 702, which extends from the center of the femoral head through the center of the knee joint, and the femoral pre-operative joint line 602. Similarly, the medial proximal tibia angle (MPTA) 708 is determined by taking the angle between a line along the tibial normal axis 704, which extends from the center of the ankle joint through the center of the knee joint, and the tibial pre-operative joint line 604. These elements are used to calculate the arithmetic hip-knee-ankle angle (aHKA) and joint line obliquity (JLO), which are integral metrics in the CPAK classification system for knee alignment in total knee arthroplasty.

The aHKA metric may be calculated by subtracting the LDFA from the MPTA, and the JLO metric may be calculated by adding the LDFA and the MPTA together. These metrics provide a detailed characterization of the knee joint alignment, which can be used to inform the selection and positioning of the implant.

These calculations may be performed based on the landmarks determined on the 3D surface of the patient's knee joint, providing a comprehensive characterization of the knee joint alignment. This characterization may be used to inform the selection and positioning of the implant, potentially leading to improved post-operative outcomes.

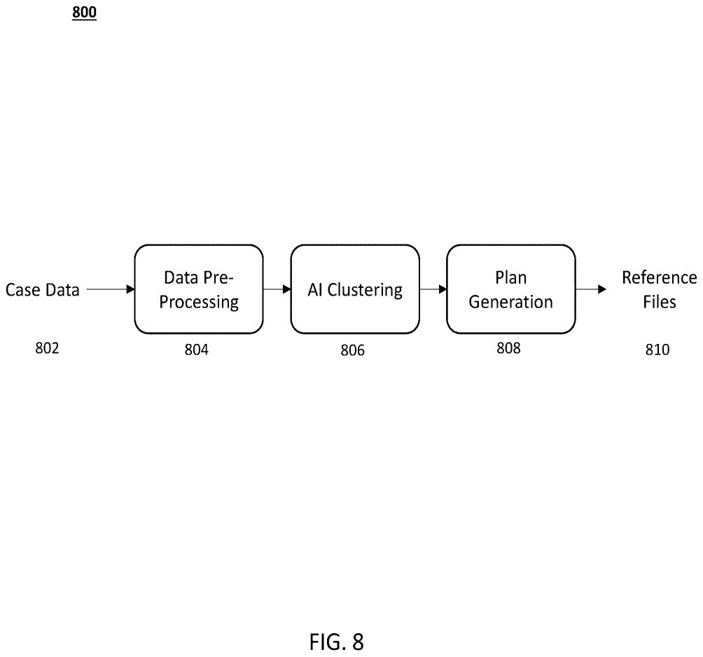

Referring now to FIG. 8, a flowchart of an AI algorithm 800 for surgical planning is depicted. The AI algorithm may be trained on surgical case data 802. The surgical case data 802 may include data from completed TKA cases, such as patient-specific anatomy data, implant selection data, and surgical planning data. The surgical case data 802 may be obtained from various sources, including but not limited to, surgical robotic systems, medical imaging systems, patient medical records, or other suitable data sources.

In some embodiments, the surgical case data 802 may be pre-processed 804. The data pre-processing 804 involves preparing the surgical case data 802 for AI clustering 806. Data pre-processing 804 may include various data processing techniques, including but not limited to, data cleaning, feature extraction, feature engineering, data normalization, and data scaling. The data pre-processing 804 may allow for the transformation of the raw case data 802 into a format that is suitable for AI clustering 806.

In certain embodiments, the AI algorithm 800 includes AI clustering 806. AI clustering 806 involves applying an AI or ML algorithm to the data (i.e., the raw case data or pre-processed data). The AI or ML algorithm may be trained to allow for the identification of surgeon-specific differences in terms of how surgeons plan TKA cases in specific patient contexts, such as preoperative deformity levels and classifications. Clustering algorithms may be unsupervised machine learning techniques that categorize data points into clusters based on their similarities. K-Means Clustering partitions data into a predetermined number of clusters by calculating the mean distance from the centroid of each cluster, making it suitable for large datasets. Hierarchical Clustering creates a hierarchy of clusters using either a bottom-up or top-down approach, ideal for smaller datasets or when the cluster structure is not predefined. DBSCAN groups points based on density, identifying outliers in low-density areas, and does not require a predefined number of clusters, allowing for the discovery of clusters with arbitrary shapes. Mean Shift Clustering identifies clusters without a predetermined number, by updating centroid candidates to the mean of points within a region. Spectral Clustering reduces dimensionality using the eigenvalues of a similarity matrix before clustering, which is beneficial for non-convex or complex cluster structures. Gaussian Mixture Models assume data points are generated from a mixture of Gaussian distributions with unknown parameters, offering flexibility to accommodate clusters of varying sizes and structures. Any known clustering algorithm, including the above referenced algorithms, may be applied. The selected algorithm selected influences data grouping and, consequently, implant selection and positioning recommendations.

Based on AI clustering 806, one or more plans may be generated 808. Plan generation 808 involves creating surgical plans based on the clustered data. These surgical plans may include recommendations for implant selection and implant positioning, which are optimized based on the CPAK classification data and the identified patterns from the AI clustering 806. A plan may be generated for each cluster and input combination. The plan values for each input combination may be averaged to generate other plans. The average may include applying weightings to particular plan values.

The plans may be compiled into reference files 810 for use by the CASS 100. The reference files 810 contain the generated surgical plans that may be used in personalized surgical planning. In some embodiments, the reference files are stored in the JSON format. Each cluster may result in a separate reference file 810.

Completed surgical procedures utilizing the AI algorithm 800 may be fed back into the algorithm for improved performance.

### Data Processing Systems for Implementing Embodiments Herein

FIG. 12 illustrates a block diagram of an exemplary data processing system 1200 in which embodiments are implemented. The data processing system 1200 is an example of a computer, such as a server or client, in which computer usable code or instructions implementing the process for illustrative embodiments of the present invention are located. In some embodiments, the data processing system 1200 may be a server computing device. For example, the data processing system 1200 may be implemented in a server or another similar computing device operably connected to a surgical system 100 as described above. The data processing system 1200 may be configured to, for example, transmit and receive information related to a patient and/or a related surgical plan with the surgical system 100.

In the depicted example, the data processing system 1200 may employ a hub architecture including a north bridge and memory controller hub (NB/MCH) 1201 and south bridge and input/output (I/O) controller hub (SB/ICH) 1202. A processing unit 1203, a main memory 1204, and a graphics processor 1205 may be connected to the NB/MCH 1201. The graphics processor 1205 may be connected to the NB/MCH 1201 through, for example, an accelerated graphics port (AGP).

In the depicted example, a network adapter 1206 connects to the SB/ICH 1202. An audio adapter 1207, a keyboard and mouse adapter 1208, a modem 1209, a read only memory (ROM) 1210, a hard disk drive (HDD) 1211, an optical drive (e.g., CD or DVD) 1212, a universal serial bus (USB) ports and other communication ports 1213, and PCI/PCIe devices 1214 may connect to the SB/ICH 1202 through a bus system 1216. The PCI/PCIe devices 1214 may include Ethernet adapters, add-in cards, and/or PC cards for notebook computers. The ROM 1210 may be, for example, a flash basic input/output system (BIOS). The HDD 1211 and the optical drive 1212 may use an integrated drive electronics (IDE) or serial advanced technology attachment (SATA) interface. A super I/O (SIO) device 1215 may be connected to the SB/ICH 1202.

An operating system may run on the processing unit 1203. The operating system may coordinate and provide control of various components within the data processing system 1200. As a client, the operating system may be a commercially available operating system. An object-oriented programming system, such as the Java™ programming system, may run in conjunction with the operating system and provide calls to the operating system from the object-oriented programs or applications executing on the data processing system 1200. As a server, the data processing system 1200 may be an IBM® eServer™ System® running the Advanced Interactive Executive operating system or the Linux operating system. The data processing system 1200 may be a symmetric multiprocessor (SMP) system that includes a plurality of processors in the processing unit 1203. Alternatively, a single processor system may be employed.

Instructions for the operating system, the object-oriented programming system, and applications or programs are located on storage devices, such as the HDD 1211, and are loaded into the main memory 1204 for execution by the processing unit 1203. The processes for embodiments described herein may be performed by the processing unit 1203 using computer usable program code, which can be located in a memory such as, for example, main memory 1204, ROM 1210, or in one or more peripheral devices.

A bus system 1216 may comprise one or more busses. The bus system 1216 may be implemented using any type of communication fabric or architecture that provides for a transfer of data between different components or devices attached to the fabric or architecture. A communication unit such as the modem 1209 or the network adapter 1206 may include one or more devices that can be used to transmit and receive data.

Those of ordinary skill in the art will appreciate that the hardware depicted in FIG. 12 may vary depending on the implementation. Other internal hardware or peripheral devices, such as flash memory, equivalent non-volatile memory, or optical disk drives may be used in addition to or in place of the hardware depicted. Moreover, the data processing system 1200 can take the form of any of a number of different data processing systems, including but not limited to, client computing devices, server computing devices, tablet computers, laptop computers, telephone or other communication devices, personal digital assistants, and the like. Essentially, data processing system 1200 can be any known or later developed data processing system without architectural limitation.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices also can “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method for planning and executing a robotic-assisted total knee arthroplasty, the method comprising:
receiving, by a processor, surface data corresponding to boney anatomy of a knee;
generating, by the processor, a three-dimensional model based on the surface data;
generating, by the processor, three-dimensional Coronal Plane Alignment of the Knee (CPAK) classification data based on the three-dimensional model; and
generating, by the processor, an optimal implant selection and implant position based on the three-dimensional CPAK classification data.

2. The method of claim 1, further comprising robotically assisting, by the processor, the total knee arthroplasty based on the optimal implant selection and implant position.

3. The method of claim 1, wherein receiving the surface data comprises receiving the surface data from a tracking system tracking a point probe.

4. The method of claim 1, wherein the three-dimensional model is further based off of an atlas model.

5. The method of claim 1, wherein generating the optimal implant selection and implant position further comprises utilizing, by the processor, a machine learning algorithm to cluster the three-dimensional CPAK classification data according to a preoperative deformity.

6. The method of claim 5, wherein the machine learning algorithm is further configured to cluster the three-dimensional CPAK classification data according to surgeon preference.

7. The method of claim 1, further comprising analyzing, by the processor, the three-dimensional model utilizing a biomechanics simulation, wherein the three-dimensional CPAK classification data is further based on the biomechanics simulation.

8. The method of claim 1, wherein the surface data is received intra-operatively.

9. The method of claim 1, wherein the three-dimensional CPAK classification data comprises data representing the knee in a plurality of flexion angles.

10. The method of claim 1, further comprising generating, by the processor, a customized patient-specific instrumentation guide based on the three-dimensional CPAK classification data.

11. A system for planning and executing a robotic-assisted total knee arthroplasty, the system comprising:
a processor configured to:
receive surface data corresponding to boney anatomy of a knee;
generate a three-dimensional model based on the surface data;
generate three-dimensional Coronal Plane Alignment of the Knee (CPAK) classification data based on the three-dimensional model; and
generate an optimal implant selection and implant position based on the three-dimensional CPAK classification data.

12. The system of claim 11, wherein the processor is further configured to robotically assist the total knee arthroplasty based on the optimal implant selection and implant position.

13. The system of claim 11, wherein the processor is further configured to receive the surface data from a tracking system tracking a point probe.

14. The system of claim 11, wherein the processor is further configured to utilize a machine learning algorithm to cluster the three-dimensional CPAK classification data according to a preoperative deformity.

15. The system of claim 14, wherein the machine learning algorithm is further configured to cluster the three-dimensional CPAK classification data according to surgeon preference.

16. The system of claim 11, wherein the processor is further configured to analyze the three-dimensional model utilizing a biomechanics simulation, wherein the three-dimensional CPAK classification data is further based on the biomechanics simulation.

17. The system of claim 11, wherein the surface data is received intra-operatively.

18. The system of claim 11, wherein the three-dimensional CPAK classification data comprises data representing the knee in a plurality of flexion angles.

19. The system of claim 11, wherein the processor is further configured to generate a customized patient-specific instrumentation guide based on the three-dimensional CPAK classification data.

20. A computer-readable medium having stored thereon instructions that, when executed by a processor, cause the processor to perform operations comprising:
receiving, by a processor, surface data corresponding to boney anatomy of a knee;
generating, by the processor, a three-dimensional model based on the surface data;
generating, by the processor, three-dimensional Coronal Plane Alignment of the Knee (CPAK) classification data based on the three-dimensional model; and
generating, by the processor, an optimal implant selection and implant position based on the three-dimensional CPAK classification data.
